(19) United States
(12) Patent Application Publication
(10) Pub. No.: US 2017/0223998 A1
(43) Pub. Date: Aug. 10, 2017

(54) FOOD SUPPLEMENT

(71) Applicant: Elena MEDEGHINI, Mazzano (IT)

(72) Inventor: Elena MEDEGHINI, Mazzano (IT)

(21) Appl. No.: 15/502,022

(22) PCT Filed: Aug. 5, 2015
(86) PCT No.: PCT/IB2015/055936

§ 371 Date: Feb. 6, 2017

(30) Foreign Application Priority Data
Aug. 6, 2014 (IT) VR2014A000200
Oct. 1, 2014 (IT) VR2014A000237

(51) Int. Cl. A23L 33/105; A23L 33/155; A61K 31/728; A23L 33/15

(52) U.S. Cl. CPC A23L 33/105; A23L 33/15; A23L 33/155; A61K 31/728; A23V 2002/00

(57) ABSTRACT

A food supplement that comprises hyaluronic acid of plant origin in the form of a sodium salt having a molecular weight comprised between 800,000 and 1,500,000.

The present invention relates to a food supplement.

Hyaluronic acid is one of the essential components of the connective tissues in the human body and other mammals, to which it gives particular properties of strength and hold of shape and structure.

A deficiency results in a weakening of the tissues, leading to the formation of wrinkles and sagging in appearance, and problems in the joints.

Its concentration in the tissues of the body tends to diminish with the onset of age. In commerce, for cosmetic use, it is typically sold under the name of sodium hyaluronate.

To partially remedy the drawbacks described above, with a targeted action in surgery and in cosmetic dermatology, injections of hyaluronic acid combined with injections of collagen proteins are used.

These injections, which in any case are painful, are now usually associated with anesthetics, and they often cause side effects such as hematoma or swelling, which may be serious in the event of allergies to and/or intolerance of the substances injected at the discretion of the medical professional together with the hyaluronic acid; in some cases, if the medical professional is not particularly expert, some unsightly effects are encountered.

The injections further ensure a localized effect which however fades in a short time and therefore forces the patient to return to the specialist periodically, with considerable costs and inconvenience.

The injections, as with creams for topical use, have high costs which rule out the possibility of its use to any users except a rich target market.

From market research it has emerged that only recently some highly specialist pharmacies, which have a customer base with high spending power and which sell expensive creams that are not within everyone's means, can, if an order is placed, obtain hyaluronic acid tablets for oral use, but without however highlighting all the beneficial properties that these can bring, since they are not present as a conventional product available at the sales point.

Such method of administration, similar to that of a medicine, is however rather difficult for all the possible users to accept, because it is proposed on the terms of a medicine and recommended by the medical professional only to patients who have serious problems in their joints.

The aim of the present invention is to make available to a huge number of users, in terms both of age and of financial means, a food supplement that is capable of preventing the problems related to the typical natural deficiency in the human body of hyaluronic acid, thus drastically eliminating the above drawbacks.

Within this aim, an object of the present invention is to provide a food supplement that is simple to use and at a very low purchase price, so that its use is advantageous from an economic viewpoint as well.

This aim and these and other objects which will become better apparent hereinafter are achieved by a food supplement according to the appended independent claims.

Further characteristics and advantages of the invention will become better apparent from the description of some preferred but not exclusive embodiments of a food supplement according to the invention.

In the embodiments illustrated, individual characteristics shown in relation to specific examples may in reality be interchanged with other, different characteristics, existing in other embodiments.

The present invention relates to a supplement for foods or for drinks.

The food supplement comprises hyaluronic acid of plant origin.

Advantageously, the supplement for foods, water and beverages can also comprise soybean isoflavones.

Conveniently the hyaluronic acid is obtained from seaweed.

Preferably, the hyaluronic acid is in the form of a sodium salt and, in particular, of sodium hyaluronate.

Conveniently, such sodium hyaluronate has a molecular weight comprised between 800,000 and 1,500,000.

Such hyaluronic acid can be administered with no contraindications every day, or it can be taken not every day but more sporadically, even with different doses and according to the habits and opportunities of the users.

As an approximate guide, one can have for example a daily administration of hyaluronic acid comprised between 75 mg and 250 mg, which can optionally also be divided into two or more administrations.

Note that if it is intended to administer 150 mg of hyaluronic acid in the form of sodium hyaluronate, then the food supplement will have to comprise a quantity equal to 171.3 mg of sodium hyaluronate.

If hyaluronic acid is associated with soybean isoflavones, then the percentage, by weight, of the soybean isoflavones with respect to the weight of the hyaluronic acid will be comprised between 0% and 46% and, preferably, between 10% and 46%.

Conveniently, the soybean isoflavones are in the form of:
Soybean (Glycine max);
Red clover (Trifolium pratense).

Conveniently, the supplement further comprises Boswellia.

Advantageously, the percentage of Boswellia with respect to hyaluronic acid is comprised between 0% and 50% and, preferably, between 5% and 50%.

Preferably, the Boswellia is in the form of:
Dry extract of Boswellia serrata;
Incense (Boswellia carterii) in powder form or as an essential oil.

Conveniently, the supplement further comprises vitamin D.

Advantageously, the percentage of vitamin D with respect to hyaluronic acid is comprised between 0% and 0.0058% and, preferably, between 0.001% and 0.0058%.

It is likewise possible for the food supplement to further comprise vitamin A.

Conveniently, the percentage of vitamin A with respect to hyaluronic acid is comprised between 0% and 0.7% and, preferably, between 0.1% and 0.7%.

In particular, the food supplement further comprises vitamin E.

Advantageously, the percentage of vitamin E with respect to hyaluronic acid is comprised between 0% and 21% and, preferably, between 2% and 21%.

According to a preferred aspect of the present invention, the invention relates to the use of a supplement for foods, water or for beverages which has the characteristics given above.

Such use involves associating the food supplement with at least one of the foods selected from the group comprising:
dairy products: milk, fresh cheese, hard cheese, grated cheese, yogurt, butter;
fruit, chopped fruit, canned fruit, vegetable cream, tomato puree, jams, syrups, nougat, preserves;
cubes for food use, concentrates in powder form, gelatins;
sweeteners: sugar, brown sugar, aspartame-based sweeteners, fructose-based sweeteners;
ice creams;
cocoa-based foods: chocolate, powdered cocoa;
coffee, tea, infusions, chamomile;
margarine, oil;
beverages, juices, water;
products based on flour, bread, etc.;
candies, confectionery products and the like.

For some elements the mixing can be done during the production process.

In other cases, in order to obtain the maximum efficacy of the hyaluronic acid without compromising the properties of the food, the use of the supplement comprises a step of mixing or solubilization of the supplement with a food, water and/or with a beverage immediately before it is consumed by the user.

This makes it possible to maintain the properties of the food and those of the food supplement unaltered.

For this reason, the food supplement can be contained in sachets, capsules or other containment devices in order to effectively preserve it and allow the convenient, fast and practical mixing or solubilization thereof with or in foods, water and/or beverages, and in order to fully ensure efficacy.

For the purposes of example, the step of mixing or solubilization of the food supplement with a food immediately before it is consumed by the user can be done with foods such as water, milk, yogurt, infusions, coffee, juices etc.

According to a further aspect, the supplement for foods or for beverages comprises soybean isoflavones.

Conveniently, the soybean isoflavones are in the form of:
Soybean (Glycine max);
Red clover (Trifolium pratense).

Conveniently, the supplement furthermore comprises Boswellia.

Preferably, the Boswellia is in the form of:
Dry extract of Boswellia serrata;
Incense (Boswellia carterii) in powder form or as an essential oil.

Conveniently, the supplement further comprises vitamin D.

It is likewise possible for the food supplement to furthermore comprise vitamin A.

In particular, the food supplement further comprises vitamin E.

According to a preferred aspect of the present invention, the invention relates to the use of a supplement for foods, for water or for beverages which has the characteristics given above.

Such use involves associating the food supplement comprising soybean isoflavones with at least one of the foods selected from the group comprising:
dairy products: milk, fresh cheese, hard cheese, grated cheese, yogurt, butter;
fruit, chopped fruit, canned fruit, vegetable cream, tomato puree, jams, syrups, nougat, preserves;
cubes for food use, concentrates in powder form, gelatins;
sweeteners: sugar, brown sugar, aspartame-based sweeteners, fructose-based sweeteners;
ice creams;
cocoa-based foods: chocolate, powdered cocoa;
coffee, tea, infusions, chamomile;
margarine, oil;
beverages, juices, water;
candies, confectionery products and the like
products based on flour, bread, etc.

For some elements the mixing can be done during the production process.

In other cases, in order to obtain the maximum efficacy of the hyaluronic acid without compromising the properties of the food, the use of the supplement comprises a step of mixing or solubilization of the supplement with a food and/or with a beverage immediately before it is consumed by the user.

This makes it possible to maintain the properties of the food and those of the food supplement unaltered.

For this reason, the food supplement can be contained in sachets, capsules or other containment devices in order to effectively preserve it and allow the convenient, fast and practical mixing or solubilization thereof with or in foods and/or beverages, and in order to fully ensure efficacy.

For the purposes of example, the step of mixing or solubilization of the food supplement with a food immediately before it is consumed by the user can be done with foods such as milk, yogurt, infusions, coffee, juices etc.

It has been found that the food supplement according to the invention can promote and increase, thanks in part to the extremely low cost, the taking, daily or otherwise, of hyaluronic acid with all the benefits that derive from it by associating it with frequently-used foods, water and beverages with a method of administration that is convenient, fast, simple, painless and without side effects.

The supplement for foods, water and/or for beverages can be made available not only at specialist pharmacies but also at other types of sales points such as supermarkets, herbalists, bars, etc.

In this manner it is no longer seen as a medicine or a privilege for a limited number of users.

Furthermore, since it is completely of plant origin it can also be taken by people who by choice or for ethical or religious reasons decide not to consume products derived from animals.

All the characteristics of the invention, indicated above as advantageous, convenient or similar, may also be missing or be substituted by equivalent characteristics.

The invention, thus conceived, is susceptible of numerous modifications and variations, all of which are within the scope of the appended claims.

In practice it has been found that in all the embodiments the invention has achieved the intended aim and objects.

In particular, it has been found that the supplement, non-invasively and thanks to the combined action of its various components, slows skin aging, limits disorders related to the menopause or andropause, and has a soothing effect.

In practice the materials employed may be any according to requirements and to the state of the art.

Moreover, all the details may be substituted by other, technically equivalent elements.

The content of Italian patent applications VR2014A000200 and VR2014A000237, the priority of which is claimed in the present application, is incorporated as a reference.

1-10. (canceled)

11. A food supplement, comprising hyaluronic acid of plant origin in the form of a sodium salt having a molecular weight comprised between 800,000 and 1,500,000.

12. The supplement according to claim 11, wherein said sodium salt comprises sodium hyaluronate.

13. The supplement according to claim 11, further comprising soybean isoflavones.

14. The supplement according to claim 13, wherein the percentage by weight of said soybean isoflavones with respect to hyaluronic acid is comprised between 10% and 46%.

15. The supplement according to claim 11, further comprising Boswellia in a percentage by weight, with respect to hyaluronic acid, comprised between 5% and 50%.

16. The supplement according to claim 11, further comprising vitamin D in a percentage by weight, with respect to hyaluronic acid, comprised between 0.001% and 0.0058%.

17. The supplement according to claim 11, further comprising vitamin A in a percentage by weight, with respect to hyaluronic acid, comprised between 0.1% and 0.7%.

18. The supplement according to claim 11, further comprising vitamin E in a percentage by weight, with respect to hyaluronic acid, comprised between 2% and 21%.

19. Use of a supplement according to claim 11, wherein it associates said supplement with at least one of the foods selected from the group comprising:
dairy products: milk, fresh cheese, hard cheese, grated cheese, yogurt, butter;
fruit, chopped fruit, canned fruit, vegetable cream, tomato puree, jams, syrups, nougat, preserves;
cubes for food use, concentrates in powder form, gelatins;
sweeteners: sugar, brown sugar, aspartame-based sweeteners, fructose-based sweeteners;
ice creams;
cocoa-based foods: chocolate, powdered cocoa;
coffee, tea, infusions, chamomile;
margarine, oil;
beverages, juices, water;
candies, confectionery products and the like;
products based on flour, bread, etc.

20. Use of a supplement according to claim 11, comprising a step of mixing or solubilization of the supplement with a food, water and/or a beverage immediately before it is consumed.
